(19) United States
(12) Patent Application Publication
(10) Pub. No.: US 2026/0016477 A1
(43) Pub. Date: Jan. 15, 2026

(54) ANTIBODY OR ANTIGEN-BINDING FRAGMENT THEREOF

(71) Applicants: SUMITOMO CHEMICAL COMPANY, LIMITED, Tokyo (JP); OSAKA PREFECTURAL HOSPITAL ORGANIZATION, Osaka-shi, Osaka (JP)

(72) Inventors: Kiyoshi HIGASHI, Osaka-shi (JP); Koichi SAITO, Osaka-shi (JP); Kazuyoshi OHKAWA, Osaka-shi (JP); Kenji IKEZAWA, Osaka-shi (JP); Norikatsu MIYOSHI, Osaka-shi (JP); Hidenori TAKAHASHI, Osaka-shi (JP)

(73) Assignees: SUMITOMO CHEMICAL COMPANY, LIMITED, Tokyo (JP); OSAKA PREFECTURAL HOSPITAL ORGANIZATION, Osaka-shi, Osaka (JP)

(21) Appl. No.: 18/993,884

(22) PCT Filed: Jul. 13, 2023
(86) PCT No.: PCT/JP2023/025857

§ 371 Date: Jan. 13, 2025

(30) Foreign Application Priority Data
Jul. 15, 2022 (JP) 2022-113671

(51) Int. Cl. G01N 33/574; C07K 16/28; G01N 33/535

(52) U.S. Cl. CPC G01N 33/57488; C07K 16/2857; G01N 33/535; G01N 33/57438; C07K 2317/20

(57) ABSTRACT

The present invention provides an antibody or an antigen-binding fragment thereof that is useful for testing for cancer, particularly early-stage cancer. The antibody or an antigen-binding fragment thereof comprises a heavy-chain CDR1 comprising the amino acid sequence of SEQ ID NO: 1, a heavy-chain CDR2 comprising the amino acid sequence of SEQ ID NO: 2, a heavy-chain CDR3 comprising the amino acid sequence of SEQ ID NO: 3, a light-chain CDR1 comprising the amino acid sequence of SEQ ID NO: 4, a light-chain CDR2 comprising the amino acid sequence represented by SAS, and a light-chain CDR3 comprising the amino acid sequence of SEQ ID NO: 6.

### TECHNICAL FIELD

The present disclosure relates to, for example, an antibody or an antigen-binding fragment thereof.

### BACKGROUND ART

Cancer is the leading cause of death in Japan, and the number of deaths from cancer is increasing year by year as the average life expectancy increases. Early diagnosis is considered to be most important for reducing the number of deaths from cancer. However, in many cases, there are no characteristic symptoms in the early stages, and at the time when a cancer is diagnosed, the cancer has already progressed, and treatment has become difficult. In addition, for cancers such as pancreatic cancer, even if the cancer tissue can be excised by surgery, the recurrence rate after surgery is high, and postoperative monitoring is required. Therefore, a simple cancer test method, such as a blood test, plays an important role since a reliable effect can be obtained by proper implementation.

So far, many antibodies against cancer antigens have been produced. In particular, monoclonal antibodies, which have high sensitivity and specificity, and can be used semi-permanently, have been commonly used. For pancreatic cancer, for which early-stage diagnosis is the most difficult, a test method using CA19-9 monoclonal antibody has been reported (NPL 1). In addition, extracellular vesicles secreted from cancer cells have been shown to be involved in cancer generation, progression, drug resistance, and metastasis (NPL 2).

### CITATION LIST

### Non-Patent Literature

NPL 1: Erxi W., Shuang Z., Kruttika B., and Qingyong M.: CA19-9 and pancreatic cancer. Clin Adv Hematol Oncol. 2013 11(1): 53-55.
NPL 2: Sunami Y., Haubler J., Zourelidis A., and Kleef J.: Cancer-associated fibroblasts and tumor cells in pancreatic cancer microenvironment and metastasis: Paracrine regulators, reciprocation and exosome. Cancers. 2022 14(3): 744.

### SUMMARY OF INVENTION

### Technical Problem

An object of the present disclosure is to provide an antibody or an antigen-binding fragment thereof that is useful for testing for cancer, in particular, early-stage cancer.

### Solution to Problem

The present inventors have conducted extensive research in order to achieve the above object and found that the object can be achieved by an antibody or an antigen-binding fragment thereof comprising a heavy-chain CDR1 comprising the amino acid sequence of SEQ ID NO: 1, a heavy-chain CDR2 comprising the amino acid sequence of SEQ ID NO: 2, a heavy-chain CDR3 comprising the amino acid sequence of SEQ ID NO: 3, a light-chain CDR1 comprising the amino acid sequence of SEQ ID NO: 4, a light-chain CDR2 comprising the amino acid sequence represented by SAS, and a light-chain CDR3 comprising the amino acid sequence of SEQ ID NO: 6. The present inventors conducted further research based on such findings and completed the present invention. Specifically, the present invention encompasses the following embodiments.

### Item 1.

An antibody or an antigen-binding fragment thereof, comprising
a heavy-chain CDR1 comprising the amino acid sequence of SEQ ID NO: 1,
a heavy-chain CDR2 comprising the amino acid sequence of SEQ ID NO: 2,
a heavy-chain CDR3 comprising the amino acid sequence of SEQ ID NO: 3,
a light-chain CDR1 comprising the amino acid sequence of SEQ ID NO: 4,
a light-chain CDR2 comprising the amino acid sequence represented by SAS, and
a light-chain CDR3 comprising the amino acid sequence of SEQ ID NO: 6.

### Item 2.

The antibody or the antigen-binding fragment thereof according to Item 1, wherein the antibody is a monoclonal antibody.

### Item 3.

The antibody or the antigen-binding fragment thereof according to Item 1, wherein the antibody is IgG.

### Item 4.

The antibody or the antigen-binding fragment thereof according to Item 1, wherein the antigen-binding fragment is Fab, F(ab′)₂, a minibody, scFv-Fc, Fv, scFv, a diabody, a triabody, or a tetrabody.

### Item 5.

A polynucleotide comprising a coding sequence of the antibody or the antigen-binding fragment thereof of any one of Items 1 to 4.

Item 6. A cell comprising the polynucleotide of Item 5.

Item 7. A test drug for cancer, comprising the antibody or the antigen-binding fragment thereof of any one of Items 1 to 4.

Item 8. The test drug according to Item 7, wherein the cancer is metastatic cancer.

Item 9. The test drug according to Item 7, wherein the cancer is pancreatic cancer.

Item 10. The test drug according to Item 7, wherein the cancer is early-stage pancreatic cancer.

Item 11. A pharmaceutical composition for diagnosing cancer, comprising the antibody or the antigen-binding fragment thereof of any one of Items 1 to 4 labeled with a diagnostic agent.

Item 12. The pharmaceutical composition according to Item 11, wherein the diagnostic agent is a radioactive substance.

Item 13. The pharmaceutical composition according to Item 11, wherein the cancer is pancreatic cancer.

Item 14. A pharmaceutical composition for treating cancer, comprising the antibody or the antigen-binding fragment thereof of any one of Items 1 to 4.

Item 15. The pharmaceutical composition for treating cancer according to Item 14, wherein the cancer is pancreatic cancer.

Item 16. A pharmaceutical composition for inhibiting metastasis of cancer, comprising the antibody or the antigen-binding fragment thereof of any one of Items 1 to 4.

Item 17. The pharmaceutical composition for inhibiting metastasis of cancer according to Item 16, wherein the cancer is pancreatic cancer.

### Advantageous Effects of Invention

According to the present disclosure, it is possible to provide an antibody or an antigen-binding fragment thereof that is useful for testing for cancer, in particular, early-stage cancer.

### BRIEF DESCRIPTION OF DRAWINGS

FIG. 1 shows clone PEMb14 that produces antibodies that react with extracellular vesicles, as demonstrated in Example 1. The left image shows the presence of one lymphocyte from observation in transmitted light. The right image shows that a lymphocyte-derived antibody reacted with extracellular vesicles from observation of immunofluorescence staining.

### DESCRIPTION OF EMBODIMENTS

In the present specification, the expressions “contain,” “comprise,” and “include” include the concepts of containing, including, consisting essentially of, and consisting of.

In the present specification, amino acid sequences are shown in single-letter codes.

### 1. Antibody or Antigen-Binding Fragment Thereof

In one embodiment, the present disclosure relates to an antibody or an antigen-binding fragment thereof comprising a heavy-chain CDR1 comprising the amino acid sequence of SEQ ID NO: 1, a heavy-chain CDR2 comprising the amino acid sequence of SEQ ID NO: 2, a heavy-chain CDR3 comprising the amino acid sequence of SEQ ID NO: 3, a light-chain CDR1 comprising the amino acid sequence of SEQ ID NO: 4, a light-chain CDR2 comprising the amino acid sequence represented by SAS, and a light-chain CDR3 comprising the amino acid sequence of SEQ ID NO: 6 (in the present specification, sometimes referred to as “the antibody or the fragment thereof of the present disclosure”). This is described below.

The antibody or the fragment thereof of the present disclosure comprises a heavy-chain variable region and a light-chain variable region. The heavy-chain variable region comprises a heavy-chain CDR1 comprising the amino acid sequence of SEQ ID NO: 1, a heavy-chain CDR2 comprising the amino acid sequence of SEQ ID NO: 2, and a heavy-chain CDR3 comprising the amino acid sequence of SEQ ID NO: 3. The light-chain variable region comprises a light-chain CDR1 comprising the amino acid sequence of SEQ ID NO: 4, a light-chain CDR2 comprising the amino acid sequence represented by SAS, and a light-chain CDR3 comprising the amino acid sequence of SEQ ID NO: 6.

The term “heavy chain” means a polypeptide chain of about 50 to 70 kDa in which an amino-terminal portion includes a variable region of about 120 to 130 or more amino acids while a carboxy-terminal portion includes a constant region. The constant region can be one of five different types, i.e., alpha (α), delta (δ), epsilon (ε), gamma (γ), and mu (μ), based on the amino acid sequence of the heavy-chain constant region. The different heavy chains differ in size; α, δ, and γ contain approximately 450 amino acids, and μ and ε contain approximately 550 amino acids. When these different types of heavy chains are combined with light chains, five well-known classes of antibodies, i.e., IgA, IgD, IgE, IgG, and IgM, including four subclasses of IgG (IgG1, IgG2, IgG3, and IgG4), are produced.

The term “light chain” means a polypeptide chain of about 25 kDa in which an amino-terminal portion includes a variable region of about 100 to 110 or more amino acids while a carboxy-terminal portion includes a constant region. The approximate length of the light chain is 211 to 217 amino acids. There are two different types, i.e., kappa (κ) and lambda (λ), based on the amino acid sequence of the constant domain.

A variable region is part of a light chain or heavy chain of an antibody, and is typically located at the amino terminus of a light chain or heavy chain, contains approximately 120 to 130 residues in a heavy chain, contains approximately 100 to 110 residues in a light chain, and is involved in binding and specificity to a specific antigen. A variable region has a wide range of sequences different among different antibodies; the variability of the sequence is concentrated in CDRs (complementarity determining regions) present in the variable region, and the portion with a low degree of variability in the variable region is referred to as a “framework region.” The CDRs in a light chain and heavy chain are mainly involved in the interactions between antibodies and antigens.

The term “CDR” means one of the three hypervariable regions (H1, H2, or H3) within the non-framework region of an antibody heavy-chain (H) variable region β-sheet framework, or one of the three hypervariable regions (L1, L2, or L3) within the non-framework region of an antibody light-chain (L) variable region R-sheet framework. The CDRs are hypervariable region sequences scattered within framework region sequences.

Preferably, the antibody or the fragment thereof of the present disclosure can comprise:
a heavy-chain variable region comprising
   (a) the amino acid sequence of SEQ ID NO: 13 or
   (b) an amino acid sequence having 85% or more identity to the amino acid sequence of SEQ ID NO: 13; and/or
a light-chain variable region comprising
   (c) the amino acid sequence of SEQ ID NO: 14 or
   (d) an amino acid sequence having 85% or more identity to the amino acid sequence of SEQ ID NO: 14.

The amino acid sequence (b) has an amino acid mutation introduced into the amino acid sequence (a). The amino acid sequence (d) has an amino acid mutation introduced into the amino acid sequence (c).

Examples of amino acid mutations include substitution, deletion, addition, and insertion. From the viewpoint that the ability of the antibody or the fragment thereof of the present disclosure to bind to an antigen is less likely to be impaired, the amino acid mutation is preferably substitution, and more preferably conservative substitution.

The term “conservative substitution” means the substitution of an amino acid residue with an amino acid residue having a similar side chain. For example, the substitution between amino acid residues having a basic side chain, such as lysine, arginine, or histidine, is considered to be a conservative substitution. The following substitutions between other amino acid residues are also considered to be a conservative substitution: the substitution between amino acid residues having an acidic side chain, such as aspartic acid or glutamic acid; the substitution between amino acid residues having an uncharged polar side chain, such as glycine, asparagine, glutamine, serine, threonine, tyrosine, or cysteine; the substitution between amino acid residues having a nonpolar side chain, such as alanine, valine, leucine, isoleucine, proline, phenylalanine, methionine, or tryptophan; the substitution between amino acid residues having a β-branched side chain, such as threonine, valine, or isoleucine; and the substitution between amino acid residues having an aromatic side chain, such as tyrosine, phenylalanine, tryptophan, or histidine.

The term “identity” of amino acid sequences refers to the degree to which two or more contrastable amino acid sequences match each other. Thus, the higher the degree of match between two amino acid sequences, the higher the identity or similarity of those sequences. The level of amino acid sequence identity is determined, for example, by using FASTA, which is a tool for sequence analysis, with default parameters. Alternatively, the level of amino acid sequence identity can be determined by using the BLAST algorithm by Karlin and Altschul (Karlin S, Altschul SF. Methods for assessing the statistical significance of molecular sequence features by using general scorings schemes, Proc Natl Acad Sci USA. 87: 2264-2268 (1990); Karlin S, Altschul SF. Applications and statistics for multiple high-scoring segments in molecular sequences, Proc Natl Acad Sci USA. 90: 5873-7 (1993)). A program called “BLASTX,” based on this BLAST algorithm, has been developed. The specific techniques of these analysis methods are known and can be found on the website of the National Center of Biotechnology Information (NCBI) (http://www.ncbi.nlm.nih.gov/). The “identity” of base sequences is also defined in the same manner as above.

The identity of the amino acid sequences (b) and (d) can each be preferably 90% or more, more preferably 95% or more, even more preferably 98% or more, and still more preferably 99% or more.

An example of the amino acid sequence (b) is (b′) an amino acid sequence having substitution, deletion, addition, or insertion (preferably substitution, more preferably conservative substitution) of one or a plurality of amino acids in the amino acid sequence of SEQ ID NO: 13.

An example of the amino acid sequence (d) is (d′) an amino acid sequence having substitution, deletion, addition, or insertion (preferably substitution, more preferably conservative substitution) of one or a plurality of amino acids in the amino acid sequence of SEQ ID NO: 14.

In both the amino acid sequences (b′) and (d′), “a plurality of” means, for example, 2 to 20, preferably 2 to 10, more preferably 2 to 5, and even more preferably 2 or 3.

The term “antibody” is used to mean monoclonal and polyclonal antibodies. Antibodies can be of any isotype, such as IgG (e.g., IgG1, IgG2, IgG3, and IgG4), IgA (e.g., IgAQ1 and IgA2), IgD, IgE, and IgM. Antibodies may be a human antibody or a non-human antibody. Examples of non-human antibodies include, but are not limited to, mouse antibodies, rat antibodies, rabbit antibodies, monkey antibodies, and chimpanzee antibodies. Antibodies may be a chimeric antibody, such as a mouse-human chimeric antibody. Antibodies can be a partially or fully humanized antibody. The antibody of the present disclosure is preferably a monoclonal antibody.

In the present specification, the antigen-binding fragment of an antibody is not particularly limited as long as it contains heavy-chain CDRs 1-3 and light-chain CDRs 1-3. Examples include Fab, F(ab′)₂, a minibody, scFv-Fc, Fv, scFv, a diabody, a triabody, and a tetrabody.

Fab contains a heavy-chain fragment containing the heavy-chain variable region and CH1 in the heavy-chain constant region, and a light chain containing the light-chain variable region and the light-chain constant region (CL), wherein the heavy-chain variable region and the light-chain variable region are associated by the non-covalent intermolecular interactions described above, or are joined by a disulfide bond. In the Fab, CH1 and CL may be disulfide-bonded between thiol groups of cysteine residues present in both CH1 and CL.

F(ab′)₂ has two pairs of the Fabs mentioned above, and each CH1 has a disulfide bond between thiol groups of cysteine residues contained in these Fabs.

Minibodies are configured such that two fragments each containing CH3 bonded to the heavy-chain variable region constituting scFV, described below, are associated by non-covalent intermolecular interactions between their CH3.

ScFv-Fc is configured such that two antibody fragments each containing scFv, described below, CH2, and CH3 are associated by non-covalent intermolecular interactions between their CH3, as with the above minibodies, and are joined by a disulfide bond between thiol groups of cysteine residues contained in each CH3.

Fv, also called the “minimum structural unit of an antibody,” is configured such that the heavy-chain variable region and the light-chain variable region are associated by non-covalent intermolecular interactions. In the Fv, thiol groups of cysteine residues present in the heavy-chain variable region and the light-chain variable region may be disulfide-bonded.

scFv is configured such that the C-terminal of the heavy-chain variable region and the N-terminal of the light-chain variable region are linked by a linker, or configured such that the N-terminal of the heavy-chain variable region and the C-terminal of the light-chain variable region are linked by a linker. scFv is also called a “single-chain antibody.”

Diabodies, triabodies, and tetrabodies are configured such that the above scFv forms dimers, trimers, and tetramers, respectively, which are associated in a structurally stable state by non-covalent intermolecular interactions between the variable regions, as with Fv etc.

The antibody or the fragment thereof of the present disclosure has an ability to bind (preferably an ability to specifically bind) to extracellular vesicles. The International Society for Extracellular Vesicles (ISEV) defines extracellular vesicles as particles that are released from cells, are delimited by a lipid bilayer, and do not contain a nucleus (cannot replicate). Based on differences in their production mechanisms, extracellular vesicles are classified into (1) exosomes (diameter: 50 to 150 nm), (2) microvesicles (diameter: 100 to 1000 nm), and (3) apoptotic vesicles (diameter: 5 μm). Known constituent protein components of extracellular vesicles include tetraspanins, such as CD9, CD63, and CD81, as well as ALIX, Tsg101, Hsp70, Hsp90, various integrins, various selectins, CD40, and Annexin V. The extracellular vesicles to which the antibody or the fragment thereof of the present disclosure has an ability to bind can be preferably extracellular vesicles released from cancer cells, more preferably extracellular vesicles released from pancreatic cancer cells, and even more preferably extracellular vesicles released from early-stage pancreatic cancer cells.

In the present specification, early-stage pancreatic cancer is stage 0 or stage 1 pancreatic cancer. In the present specification, the cancer stages are classified according to the UICC, 8th edition.

The antibody or the fragment thereof of the present disclosure may be bound or fused to other peptides, oligopeptides, or proteins. Examples of other peptides, oligopeptides, or proteins include albumin (e.g., serum albumin), protein tags (e.g., biotin, His tag, FLAG tag, Halo tag, MBP tag, HA tag, Myc tag, V5 tag, and PA tag), fluorescent proteins (e.g., GFP, Azami-Green, ZsGreen, GFP2, HyPer, Sirius, BFP, CFP, Turquoise, Cyan, TFP1, YFP, Venus, ZsYellow, Banana, KusabiraOrange, RFP, DsRed, AsRed, Strawberry, JRed, KillerRed, Cherry, HcRed, and mPlum), luminescent proteins (e.g., luciferase, β-galactosidase, chloramphenicol acetyltransferase, and β-glucuronidase), secretory signal sequences (e.g., Igκ signal sequence), protease recognition sequences (e.g., TEV protease recognition sequence), expression-enhancing sequences, solubilization sequences, and multimerization domains (e.g., cartilage oligomeric matrix protein domain, leucine zipper domain, collagen-like domain, cholera toxin B subunit domain, tetrabrachion coiled core domain, reovirus protein σ1 domain, and hepatitis delta antigen domain). When the antibody or the fragment thereof of the present disclosure is bound or fused to a multimerization domain, an additional antibody or antigen-binding fragment thereof of the same type as, or a different type from, the antibody or the fragment thereof of the present disclosure may be further bound or fused to the multimerization domain to form a multimer (homomultimer or heteromultimer).

The antibody or the fragment thereof of the present disclosure may be chemically modified as long as its ability to bind to an antigen is not significantly impaired.

The antibody or the fragment thereof of the present disclosure may have a carboxyl group (—COOH), a carboxylate (—COO—), an amide group (—CONH₂), or an ester group (—COOQ) at the C-terminus. “Q” in the ester group is, for example, a C₁₋₆ alkyl group, such as methyl, ethyl, n-propyl, isopropyl, or n-butyl; a C₃₋₈ cycloalkyl group, such as cyclopentyl or cyclohexyl; a C₆₋₁₂ aryl group, such as phenyl or α-naphthyl; a phenyl-C₁₋₂ alkyl group, such as benzyl or phenethyl; a C₇₋₁₄ aralkyl group including an α-naphthyl-C₁₋₂ alkyl group, such as α-naphthylmethyl; and a pivaloyloxymethyl group.

The antibody or the fragment thereof of the present disclosure may have an amidated or esterified carboxyl group (or carboxylate), which is not the carboxyl group at the C-terminus. As the ester in this case, for example, the C-terminal ester mentioned above or the like is used.

The antibody or the fragment thereof of the present disclosure also includes those in which the amino group of the N-terminal amino acid residue is protected by a protecting group (e.g., a C₁₋₆ acyl group, such as C₁₋₆ alkanoyl, such as a formyl group or an acetyl group); those in which the N-terminal glutamine residue that can be produced by cleavage in vivo is converted to pyroglutamic acid; those in which a substituent (e.g., —OH, —SH, an amino group, an imidazole group, an indole group, or a guanidino group) on an amino acid side chain in the molecule is protected by an appropriate protecting group (e.g., a C₁₋₆ acyl group, such as C₁₋₆ alkanoyl, such as a formyl group or an acetyl group); conjugated proteins such as those called “glycoproteins,” to which a sugar chain is bound; and the like.

The antibody or the fragment thereof of the present disclosure can be produced, for example, by a method comprising a step of culturing a host transformed with a polynucleotide comprising the coding sequence of the antibody or the fragment thereof of the present disclosure (the polynucleotide of the present disclosure), and collecting a fraction comprising the antibody of the present disclosure.

The polynucleotide can be a nucleotide in the form of a polymer of any length that is either deoxyribonucleotide or ribonucleotide or an analog thereof. The sequence of a polynucleotide is composed of four types of nucleotide bases: adenine (A), cytosine (C), guanine (G), thymine (T), and uracil (U) instead of thymine when the polynucleotide is an RNA. Examples of polynucleotides can include genes, gene fragments, exons, introns, messenger RNAs (mRNAs), transfer RNAs, ribosomal RNAs, ribozymes, cDNAs, and recombinant polynucleotides. A polynucleotide also refers to both double-stranded and single-stranded molecules. Additionally, the polynucleotide may have the following chemical modifications described as examples. Specifically, to prevent degradation by hydrolases, such as nucleases, the phosphate residue (phosphate) of each nucleotide can be substituted with a chemically modified phosphate residue, such as phosphorothioate (PS), methylphosphonate, or phosphorodithionate. The hydroxyl group at position 2 of the ribose of each ribonucleotide may also be substituted with —OR (R represents, for example, —CH₃, —CH₂CH₂OCH₃, —CH₂CH₂NHC(NH))NH₂, —CH₂CONHCH₃, or —CH₂CH₂CN). Additionally, the base moiety (pyrimidine, purine) may be chemically modified by, for example, introduction of a methyl group or a cationic functional group into position 5 of the pyrimidine base, or substitution of the carbonyl group at position 2 with thiocarbonyl. Additionally, the phosphate moiety or the hydroxyl moiety may be modified with, for example, biotin, an amino group, a lower alkyl amine group, or an acetyl group. The modifications are not limited to those described here.

The polynucleotide of the present disclosure is not particularly limited as long as it comprises the coding sequence of the antibody or the fragment thereof of the present disclosure. Preferably, the polynucleotide of the present disclosure comprises the coding sequence in a state that enables expression of the antibody or the fragment thereof of the present disclosure. The polynucleotide of the present disclosure may comprise other sequences in addition to the coding sequence. Other sequences include a secretory signal peptide coding sequence, a promoter sequence, an enhancer sequence, a repressor sequence, an insulator sequence, an origin of replication, a drug-resistance-gene-coding sequence, and the like, which are located adjacent to the coding sequence of the antibody of the present disclosure. The polynucleotide of the present disclosure may be a linear polynucleotide or a circular polynucleotide (e.g., a vector).

Specific examples of the polynucleotide of the present disclosure include (I) a polynucleotide comprising a base sequence encoding at least one member selected from the group consisting of the heavy chain, heavy-chain variable region, and heavy-chain CDRs 1 to 3 of the antibody or the fragment thereof of the present disclosure; (II) a polynucleotide comprising a base sequence encoding at least one member selected from the group consisting of the light chain, light-chain variable region, and light-chain CDRs 1 to 3 of the antibody or the fragment thereof of the present disclosure; and (III) a combination of a polynucleotide comprising a base sequence encoding at least one member selected from the group consisting of the heavy chain, heavy-chain variable region, and heavy-chain CDRs 1 to 3 of the antibody or the fragment thereof of the present disclosure and a polynucleotide comprising a base sequence encoding at least one member selected from the group consisting of the light chain, light-chain variable region, and light-chain CDRs 1 to 3 of the antibody or the fragment thereof of the present disclosure.

The polynucleotide of the present disclosure can be chemically synthesized, or can also be obtained by PCR amplification using synthetic primers that can hybridize to the 3′ and 5′ ends of a sequence, or by cloning using oligonucleotide probes specific for a particular nucleic acid sequence, from a suitable source (for example, cDNA isolated from antibody-expressing cells, such as hybridoma cells, selected to express an antibody). The amplified nucleic acid produced by PCR can then be cloned into a replicable cloning vector using any method well known in the art.

When the polynucleotide of the present disclosure is obtained, a vector for producing the antibody or the fragment thereof can be produced by recombinant DNA technology using a method well known in the art, and an expression vector containing the coding sequence of the antibody or the fragment thereof and appropriate transcription and translation control signals can be further constructed using a well-known method. In addition, although the constant region differs greatly between species, such as humans and rabbits, the constant regions of antibodies of the same species are very similar. Thus, antibodies of humans, rabbits, and the like can be produced by cloning into a vector containing a nucleotide sequence encoding the constant region of their species.

The expression vector can be introduced into host cells by a conventional method, and then the transfected cells can be cultured by a conventional method to produce the antibody or the fragment thereof of the present disclosure. In order to express the entirety of an immunoglobulin molecule, a vector encoding both a heavy chain and a light chain may be co-expressed in host cells.

Various host-expression vector systems can be used to express the antibody or the fragment thereof of the present disclosure. Examples of host-expression vector systems include microorganisms, such as bacteria (for example, Escherichia coli, Bacillus subtilis) transformed with a recombinant bacteriophage DNA, plasmid DNA, or cosmid DNA expression vector containing the antibody coding sequence, and yeast (for example, Saccharomyces pikia) transformed with a recombinant yeast expression vector containing the antibody coding sequence, insect cells infected with a recombinant virus expression vector (for example, baculovirus) containing the antibody coding sequence, plant cells infected with a recombinant virus expression vector (for example, cauliflower mosaic virus, TMV) or transformed with a recombinant plasmid expression vector (for example, Ti plasmid) containing the antibody coding sequence, and mammalian cells (for example, COS, CHO, HEK293, 3T3 cells) having a recombinant expression construct containing a promoter derived from the genome of mammalian cells (for example, metallothionein promoter) or a promoter derived from a mammalian virus (for example, late adenovirus promoter).

When the antibody or the fragment thereof of the present disclosure is produced by recombinant expression, the antibody or the fragment thereof can be purified by a method known in the art for purifying immunoglobulin molecules, such as chromatography (for example, ion exchange chromatography, protein A chromatography, gel filtration column chromatography), centrifugation, or salting out. Furthermore, the antibody or the fragment thereof of the present disclosure can be fused with a known heterologous polypeptide sequence in order to facilitate purification. For example, the antibody or the fragment thereof of the present disclosure can be purified by recombinantly adding a poly-histidine tag (His tag), FLAG tag, hemagglutinin tag (HA tag), or myc tag.

### 2. Cells

In one embodiment, the present disclosure relates to a cell comprising the polynucleotide of the present disclosure.

The cell encompasses, for example, cells transduced with the polynucleotide of the present disclosure.

Preferably, the cell is an isolated cell. The cell may be a cultured cell, and may be a primary cultured cell or subcultured cell. The cell may be a cell line. The cell may also be an iPS cell.

Examples of the cell include colon bacteria, such as Escherichia coli K12; Bacillus bacteria, such as Bacillus subtilis MI114; yeasts, such as Saccharomyces cerevisiae AH22; and insect cells and animal cells, such as Spodoptera frugiperda-derived Sf cell line, Trichoplusia ni-derived High Five cell line, and olfactory nerve cells. Preferred examples of animal cells include mammal-derived cultured cells, such as COS7 cells, CHO cells, HEK293 cells, HEK293FT cells, Hela cells, PC12 cells, N1E-115 cells, and SH-SY5Y cells.

### 3. Test Drug for Disease

The test drug for a disease of the present disclosure is characterized by comprising the antibody or the fragment thereof of the present disclosure. The test drug of the present disclosure is a drug for testing for the possibility of having a cancer, in particular, an early-stage cancer.

The test drug for a disease of the present disclosure can be used for a method for testing for (or determining) the possibility of having a cancer, in particular, an early-stage cancer, which comprises a step of measuring the amount or concentration of an antigen that binds to the antibody or the fragment thereof of the present disclosure in a biological sample collected from a subject (in the present specification, the method is sometimes referred to as “the test method of the present disclosure”). This will be described below.

The cancers to be tested are not particularly limited and include cancers of all types, stages, and the like. The types of cancers are preferably pancreatic cancer, and particularly preferably early-stage pancreatic cancer (stage 0 or stage 1). Examples of the types of pancreatic cancer include pancreatic ductal cancer, pancreatic endocrine tumor, intraductal papillary mucinous neoplasm, mucinous cystic neoplasm, and acinar cell carcinoma. Preferred is pancreatic ductal cancer. The cancers to be tested can be metastatic cancer. When the primary cancer is pancreatic cancer, examples of the metastasis sites include the liver, peritoneum, lungs, lymph nodes, and bones.

The subject is a target organism of the test method of the present disclosure, and the biological species is not particularly limited. Examples of the biological species of the subject include various mammals, such as humans, monkeys, rabbits, mice, rats, dogs, and cats, and humans are preferable.

The condition of the subject regarding cancer is not particularly limited. Examples of the subject include a subject in which whether a cancer has been developed is unknown, a subject who has no history of cancer, a subject who has a history of cancer and has been treated for the cancer, and a subject who has been already determined to have a cancer (or not have a cancer) by another determination method.

The biological sample is not particularly limited as long as the biological sample can contain an antigen that binds to the antibody or the fragment thereof of the present disclosure. Examples of the biological sample include body fluids, such as whole blood, serum, plasma, saliva, spinal fluid, synovial fluid, urine, tissue fluid, sweat, and tears, and samples derived from these body fluids. The samples derived from the body fluids are not particularly limited as long as the samples are prepared from the body fluids, and examples include a sample obtained from a body fluid by, for example, concentrating or purifying an antigen to which the antibody or the fragment thereof of the present disclosure binds. Preferable examples of the body fluids include whole blood, serum, and plasma. The biological samples may be used alone or in a combination of two or more.

The biological sample can be collected from the subject by a method known to those skilled in the art. For example, whole blood can be collected by blood collection using a syringe or the like. The blood collection is preferably performed by medical staff, such as a doctor and a nurse. Serum is a portion of blood obtained by removing blood cells and specific blood coagulation factors from the blood, and can be obtained, for example, as a supernatant after coagulation of blood. Plasma is a portion of blood obtained by removing blood cells from the blood, and can be obtained, for example, as a supernatant when blood is subjected to centrifugation under conditions in which blood is not coagulated.

The test method of the present disclosure comprises a step of measuring the amount or concentration of the antigen that binds to the antibody or the fragment thereof of the present disclosure, and the method is not particularly limited. Examples of the method include immunoassays. Immunoassays can be widely used regardless of whether a direct method, an indirect method, a uniform method, a non-uniform method, a competitive method, a non-competitive method, and the like. More specific examples of immunoassays include ELISA (direct method, indirect method, sandwich method, competitive method, etc.), radioimmunoassay (RIA), immunoradiometric assay (IRMA), enzyme immunoassay (EIA), sandwich EIA, immunochromatography, western blotting, immunoprecipitation, slot or dot blot assay, immunohistochemical staining, fluorescent immunoassay, immunoassay using avidin-biotin or streptavidin-biotin system, and immunoassay using a surface plasmon resonance (SPR) method.

Since the antibody or the fragment thereof of the present disclosure binds to extracellular vesicles, the amount or concentration of the extracellular vesicles that constitute the antigen of the antibody of the present disclosure can be measured by combining with an antibody or a fragment thereof against the constituent components of extracellular vesicles. Examples of such test methods include a sandwich method, ExoCounter, and exosome sensing chips. The detection methods may be used alone or in a combination of two or more.

In the step of measuring the amount or concentration of the antigen that binds to the antibody or the fragment thereof of the present disclosure, the type of label for a labeled object to be used for detecting the antigen (for example, a labeled antibody) is not particularly limited. Examples of the label include fluorescent substances, luminescent substances, pigments, enzymes, colloidal gold, and radioisotopes. Among them, enzyme labels, such as peroxidase and alkaline phosphatase, are more preferable from the viewpoint of safety, economy, detection sensitivity, etc.

The method for measuring and calculating the amount or concentration of the antigen that binds to the antibody or the fragment thereof of the present disclosure will be described below in detail.

An example of one embodiment of the method includes a step of bringing the antibody or the fragment thereof of the present disclosure into contact with a biological sample collected from a subject, and a step of measuring the amount of the antigen bound to the antibody or the fragment thereof of the present disclosure.

The mode of contact with the biological sample collected from a subject is not particularly limited, and an appropriate mode can be selected according to the type of the method (for example, various immunoassays) for measuring the amount or concentration of the antigen that binds to the antibody or the fragment thereof of the present disclosure.

Examples of the contact mode include a mode in which the antibody or the fragment thereof of the present disclosure and the biological sample are brought into contact with each other in a state in which only one of the antibody or the fragment thereof of the present disclosure and the biological sample is fixed to a solid phase, and a mode in which the antibody or the fragment thereof of the present disclosure and the biological sample are brought into contact with each other in a state in which neither the antibody or the fragment thereof of the present disclosure nor the biological sample is fixed to a solid phase. Among them, from the viewpoint of efficiency etc., a preferable mode is a mode in which the antibody or the fragment thereof of the present disclosure and the biological sample are brought into contact with each other in a state in which only the antibody or the fragment thereof of the present disclosure is fixed to a solid phase.

When only one of the antibody or the fragment thereof of the present disclosure and the biological sample is fixed to a solid phase, after the fixation, the solid phase is preferably washed with a solution containing tris hydroxymethylaminomethane (Tris) and/or an ether-type nonionic surfactant.

The solid phase is not particularly limited as long as the antibody or the fragment thereof of the present disclosure and the biological sample can be fixed to the solid phase. Examples of the solid phase include plates, slides, and membranes containing polystyrene, glass, nitrocellulose, or the like as a main component. The solid phase may be coated with a component for more easily fixing the antibody or the fragment thereof of the present disclosure and the biological sample, such as a readily reactive compound (a compound having a readily reactive group, colloidal gold, etc.). Examples of the compound having a readily reactive group include compounds having a group capable of forming a covalent bond with a sugar chain or a sugar chain derivative, such as a (1H-imidazol-1-yl)carbonyl group, a succinimidyloxycarbonyl group, an epoxy group, an aldehyde group, an amino group, a thiol group, a carboxyl group, an azide group, a cyano group, an active ester group (1H-benzotriazole-1-yloxycarbonyl group, pentafluorophenyloxycarbonyl group, paranitrophenyloxycarbonyl group, etc.), and a carbonyl halide group (carbonyl chloride group, carbonyl fluoride group, carbonyl bromide group, and carbonyl iodide group).

Examples of the compound having a readily reactive group include epoxysilane and polylysine.

When a solid phase coated with a readily reactive compound is used, the readily reactive compound is preferably blocked using a buffer solution containing bovine serum albumin (BSA) or the like. The blocking time is preferably 60 minutes or longer. The blocking solution preferably contains tris hydroxymethylaminomethane (Tris) and/or an ether-type nonionic surfactant.

The mode for measuring the amount of the antigen bound to the antibody or the fragment thereof of the present disclosure is not particularly limited, and an appropriate mode can be selected according to the type of the method described above (for example, various immunoassays) for measuring the amount or concentration of the antigen that binds to the antibody or the fragment thereof of the present disclosure. The measurement can be performed, for example, by quantifying a signal derived from the label of a labeled object used. As a more specific mode, the measurement can be performed, for example, by bringing the labeled antibody or the fragment thereof of the present disclosure or the labeled antibody against the antigen into contact with the antigen bound to the antibody or the fragment thereof of the present disclosure, and quantifying the signal derived from the label of the bound labeled antibody.

Based on the obtained signal amount, the amount of the antigen bound to the antibody or the fragment thereof of the present disclosure can be calculated. For example, in the case of a non-competitive method, the obtained signal amount can be used as is as the amount of the antigen bound to the antibody or the fragment thereof of the present disclosure. In addition, as another example, in the case of a competitive method, since the obtained signal amount and the amount of the antigen bound to the antibody or the fragment thereof of the present disclosure are inversely proportional to each other, the amount of the antigen bound to the antibody or the fragment thereof of the present disclosure can be calculated from a signal amount obtained based on this relationship.

According to the test method of the present disclosure, it is possible to provide a value of the antigen that binds to the antibody or the fragment thereof of the present disclosure, which is an index for detecting development of a cancer, such as pancreatic cancer, whereby it is possible to assist in determining the possibility of having a cancer, such as pancreatic cancer.

In one embodiment, the test method of the present disclosure preferably further comprises a step of determining that the possibility of having a cancer, such as pancreatic cancer, in the subject is high when the measured value of the amount or concentration of the antigen that binds to the antibody or the fragment thereof of the present disclosure is equal to or higher than a preset cutoff value. Here, the “possibility of having a cancer, such as pancreatic cancer” means the “possibility of having a cancer, such as pancreatic cancer, at the time when the biological sample is collected.”

According to the test method of the present disclosure, it is possible to determine the possibility of having a cancer. In addition, according to the test method of the present disclosure, the possibility of having a cancer can be determined with higher sensitivity, and thus a subject truly having a cancer can be more reliably determined to have a cancer (that is, the possibility of erroneously determining that the subject does not have a cancer can be further reduced) by the test method of the present disclosure.

The cutoff value can be set as appropriate by those skilled in the art from the viewpoint of sensitivity, specificity, positive predictive value, negative predictive value, etc., and can be, for example, the average value, the percentile value, or the maximum value of the amount or concentration values of the antigen that binds to the antibody or the fragment thereof of the present disclosure in biological samples collected from subjects who do not have a cancer. More specifically, for example, the cutoff value can be set by measuring the amount or concentration of the antigen that binds to the antibody or the fragment thereof of the present disclosure in a biological sample collected from both subjects who do not have a cancer and subjects who have a cancer, such as pancreatic cancer, and performing statistical analysis based on, for example, analysis of a receiver operating characteristic (ROC) curve using the measured values (more specifically, for example, a method using a Youden index).

The cutoff value can also be the amount or concentration value of the antigen that binds to the antibody or the fragment thereof of the present disclosure in a biological sample collected from the same subject before a certain period of time. The “certain period of time” is not particularly limited as long as the certain period of time is a period in which the amount or concentration of the antigen that binds to the antibody or the fragment thereof of the present disclosure can change within the same subject. Examples of the certain period of time include periods of about 1 month to 10 years, about 2 months to 5 years, about 3 months to 2 years, and about 4 months to 1 year.

A modification of the test method of the present disclosure includes a method comprising a step of determining that the subject is likely to develop a cancer, such as pancreatic cancer, in the future, when the amount or concentration value of the antigen that binds to the antibody or the fragment thereof of the present disclosure is higher than the amount or concentration value of the antigen that binds to the antibody or the fragment thereof of the present disclosure in a biological sample collected from the same subject before a certain period of time. The risk of developing cancer in the future is determined by the test method comprising this step.

The “certain period of time” is not particularly limited as long as the certain period of time is a period in which the amount or concentration of the antigen that binds to the antibody or the fragment thereof of the present disclosure can change within the same subject. Examples of the certain period of time include periods of about 1 month to 10 years, about 2 months to 5 years, about 3 months to 2 years, and about 4 months to 1 year.

The degree of “higher” is not particularly limited, and examples include the amount or concentration value of the antigen that binds to the antibody or the fragment thereof of the present disclosure being 2 times or more, 4 times or more, 8 times or more, and 20 times or more the amount or concentration value of the antigen that binds to the antibody or the fragment thereof of the present disclosure in a biological sample collected from the same subject before the certain period of time.

When it is determined by the test method of the present disclosure that the possibility of having a cancer, such as pancreatic cancer, in the subject is high, development of a cancer, such as pancreatic cancer, can be diagnosed with higher accuracy by further combining the test method of the present disclosure with another diagnostic method. The other diagnostic method is not particularly limited, and various known diagnostic methods can be used. Examples of the diagnostic method include a biopsy method, a PET test method, a CT test method, an ultrasonic test method, and a tumor marker test method. Among them, from the viewpoint of being able to diagnose pancreatic cancer with higher accuracy, a biopsy method, a PET test method, a CT test method, an ultrasonic test method, and the like are preferable. These diagnostic methods may be used alone or in a combination of two or more.

The test drug of the present disclosure may be in the form of a composition comprising the antibody or the fragment thereof of the present disclosure. The composition may also comprise other components, if necessary. Examples of the other components include a base, a carrier, a solvent, a dispersant, an emulsifier, a buffer, a stabilizer, an excipient, a binder, a disintegrant, a lubricant, a thickener, a moisturizer, a colorant, a flavor, and a chelating agent.

The test drug of the present disclosure may be in the form of a kit comprising the antibody or the fragment thereof of the present disclosure. The kit may comprise instruments, reagents, and the like that can be used to carry out the test method of the present disclosure.

Examples of the instruments include a test tube, a microtiter plate, agarose particles, latex particles, a column for purification, an epoxy-coated slide glass, and a colloidal gold-coated slide glass.

Examples of the reagents include an antibody against the antigen of the antibody or the fragment thereof of the present disclosure or a labeled antibody thereof, and standard samples (positive control, negative control).

The antibody against the antigen of the antibody or the fragment thereof of the present disclosure may be an antibody against a molecule (for example, protein) to which the antibody of the present disclosure binds.

The type of the label for producing the labeled antibody is not particularly limited. Examples of the label include fluorescent substances, luminescent substances, pigments, enzymes, colloidal gold, and radioisotopes. Among them, enzyme labels, such as peroxidase and alkaline phosphatase, are preferable from the viewpoint of safety, economy, detection sensitivity, etc.

The standard sample may be the antigen of the antibody or the fragment thereof of the present disclosure. The antigen of the antibody or the fragment thereof of the present disclosure can be, for example, isolated and purified from a biological sample, such as extracellular vesicles secreted by cancer cells, by immunoprecipitation using the antibody or the fragment thereof of the present disclosure.

The present disclosure also provides a pharmaceutical composition for diagnosing a cancer, such as pancreatic cancer, comprising the antibody or the fragment thereof of the present disclosure labeled with a diagnostic agent. When it is determined by the test method of the present disclosure that the possibility of having a cancer, such as pancreatic cancer, in the subject is high, development of a cancer, such as pancreatic cancer, can be diagnosed with higher accuracy by, for example, intravascularly, intramuscularly, subcutaneously, or intraperitoneally administering the pharmaceutical composition comprising the antibody or the fragment thereof of the present disclosure labeled with the diagnostic agent, and examining the whole body.

The pharmaceutical composition for diagnosing a disease of the present disclosure can further comprise a pharmaceutically acceptable carrier. Examples of usable pharmaceutically acceptable carriers include standard pharmaceutical carriers known in the art, such as phosphate-buffered saline solutions, water, oil/water emulsions and other emulsions, and various types of wetting agents. In addition, other components, such as a pharmaceutical-grade stabilizer, buffer, preservative, and excipient, can also be used in the pharmaceutical composition for diagnosing a disease of the present disclosure. Preparation of the pharmaceutical composition in consideration of pH, isotonicity, stability, etc. can be carried out by a known method.

Examples of the diagnostic agent for labelling the antibody or the fragment thereof of the present disclosure include radioactive substances, fluorescent materials, luminescent materials, bioluminescent materials, and photoacoustic imaging materials. Examples of radioactive substances include various positron-emitting metals, such as zirconium (⁸⁹Zr), iodine (¹³¹I, ¹²⁵I, ¹²⁴I, ¹²³I, and ¹²¹I), indium (¹¹⁵In, ¹¹³In, ¹¹²In, and ¹¹¹In), technetium (⁹⁹Tc), tallium (²⁰¹Ti), and gallium (⁶⁸Ga, ⁶⁷Ga). Examples of luminescent materials include umbelliferone, fluorescein, and fluorescein isothiocyanate. Examples of luminescent materials include luminol. Examples of bioluminescent materials include luciferase, luciferin, and aequorin. Examples of photoacoustic imaging materials include gold nanoparticles, single-layer carbon nanotubes, indocyanine green, and methylene blue.

Examples of the method for labeling the antibody or the fragment thereof of the present disclosure include a method of indirectly binding a compound using a linker, such as polyethylene glycol, and a method of directly binding a compound by forming a disulfide bond at a cysteine residue of the antibody or the fragment thereof. It is also possible to directly bind a compound to the antibody or the fragment thereof using a heterologous bifunctional crosslinking agent, such as N-succinyl-3-(2-pyridyldithio)propionate. Furthermore, a compound can also be bound by oxidizing the sugar chain in the Fc region of the antibody.

The dose of the antibody or the fragment thereof labeled with the diagnostic agent, which is contained in the pharmaceutical composition for diagnosing a cancer, such as pancreatic cancer, of the present disclosure is not particularly limited as long as the dose is a pharmacologically effective amount. The dose can be determined as appropriate according to race, gender, age, type of cancer, etc., and is usually 0.01 to 1,000 mg/kg and preferably 0.1 to 100 mg/kg. The time required to preferentially concentrate the antibody or the fragment thereof of the present disclosure labeled with the diagnostic agent on the binding site in the subject, and to remove the antibody or the fragment thereof not bound to the binding site to the background level can be determined as appropriate according to the type of the diagnostic agent to be used, the administration method, etc., and is, for example, about 6 to 48 hours after administration. In addition, when the disease is monitored, the disease is repeatedly monitored, for example, at a time interval of 5 to 20 days after administration for 1 to 12 months from the first diagnosis.

The presence of the antibody or the fragment labeled with the diagnostic agent contained in the pharmaceutical composition for diagnosing a disease of the present disclosure can be detected by examining the whole body of the subject using a known method. The detection method depends on the type of the diagnostic agent to be used, and examples of methods that can be used in the present disclosure include computed tomography, positron emission tomography, magnetic resonance imaging, ultrasonography, and photoacoustic imaging.

Examples of diseases that can be diagnosed using the pharmaceutical composition of the present disclosure include cancers, such as the pancreatic cancers described above, and the pharmaceutical composition of the present disclosure is particularly useful for diagnosing a cancer that highly expresses the antigen of the antibody or the fragment thereof of the present disclosure. That is, when it is determined by the test method of the present disclosure that the possibility of having a cancer, such as pancreatic cancer, in the subject is high, it is possible to diagnose a cancer, such as pancreatic cancer, in the subject with higher accuracy by administering the pharmaceutical composition of the present disclosure.

### 4. Cancer Treatment

When it is determined by the test method of the present disclosure that the possibility of having a cancer, such as pancreatic cancer, in the subject is high, or when the subject is diagnosed as having a cancer, such as pancreatic cancer, by the test method of the present disclosure in combination with another diagnostic method, it is made possible to treat the subject's cancer by performing cancer treatment on the subject. In addition, according to the test method of the present disclosure, the possibility of having a cancer, such as pancreatic cancer, can be determined with higher sensitivity, and so a subject truly having a cancer can be treated more reliably by the test method of the present disclosure or by a combination with another diagnostic method (that is, the possibility of excluding the subject truly having a cancer from subjects to be treated can be reduced).

The method of the cancer treatment is not particularly limited, and various known treatment methods can be used. Examples of the treatment method include chemotherapy, surgical treatment, radiotherapy, and immunotherapy. These treatments can be carried out according to known methods.

The therapeutic agent used for chemotherapy is not particularly limited, and various anticancer agents can be used. Examples of anticancer agents include alkylating agents, antimetabolites, microtubule inhibitors, antibiotic anticancer agents, topoisomerase inhibitors, platinum preparations, molecular target drugs, hormone agents, and biological preparations. Examples of the alkylating agents include cyclophosphamide, ifosfamide, nitrosourea, dacarbazine, temozolomide, nimustine, busulfan, melphalan, procarbazine, and ranimustine. Examples of the antimetabolites include enocitabine, carmofur, capecitabine, tegafur, tegafur-uracil, tegafur-gimeracil-oteracil potassium, gemcitabine, cytarabine, cytarabine ocfosfate, nelarabine, fluorouracil, fludarabine, pemetrexed, pentostatin, methotrexate, cladribine, doxifluridine, hydroxycarbamide, and mercaptopurine. Examples of the microtubule inhibitors include alkaloid-based anticancer agents, such as vincristine, and taxane-based anticancer agents, such as docetaxel and paclitaxel. Examples of the antibiotic anticancer agents include mitomycin C, doxorubicin, epirubicin, daunorubicin, bleomycin, actinomycin D, aclarubicin, idarubicin, pirarubicin, peplomycin, mitoxantrone, amrubicin, and zinostatin stimalamer. Examples of the topoisomerase inhibitors include CPT-11, irinotecan, and nogitecan having topoisomerase I inhibitory action, and etoposide and sobuzoxane having topoisomerase II inhibitory action. Examples of the platinum preparations include cisplatin, nedaplatin, oxaliplatin, and carboplatin. Examples of the hormone agents include dexamethasone, finasteride, tamoxifen, anastrozole, exemestane, ethinylestradiol, chlormadinone, goserelin, bicalutamide, flutamide, prednisolone, leuprorelin, letrozole, estramustine, toremifene, fosfestrol, mitotane, methyltestosterone, medroxyprogesterone, and mepitiostane. Examples of the biological preparations include immune checkpoint inhibitors, such as anti-PD-1 antibody and anti-PD-L1 antibody, interferons α, β, and γ, interleukin 2, ubenimex, and dried BCG. Examples of the molecular target drugs include rituximab, alemtuzumab, trastuzumab, cetuximab, panitumumab, imatinib, dasatinib, nilotinib, gefitinib, erlotinib, temsirolimus, bevacizumab, VEGF trap, sunitinib, sorafenib, tocilizumab, bortezomib, gemtuzumab-ozogamicin, ibritumomab-ozogamicin, ibritumomab tiuxetan, tamibarotene, and tretinoin. In addition to the molecular target drugs specified here, the following molecular target drugs may be included: inhibitors targeting angiogenesis, such as human epidermal growth factor receptor 2 inhibitor, epidermal growth factor receptor inhibitor, Bcr-Abl tyrosine kinase inhibitor, epidermal growth factor tyrosine kinase inhibitor, mTOR inhibitor, and endothelial growth factor receptor 2 inhibitor (α-VEGFR-2 antibody); tyrosine kinase inhibitors, such as MAP kinase inhibitor; inhibitors targeting cytokine, proteasome inhibitors, and antibody-anticancer agent formulations. These inhibitors also include antibodies.

Typical therapeutic agents for pancreatic cancer include gemcitabine, TS-1, erlotinib, a concomitant drug of gemcitabine and erlotinib, a concomitant drug of gemcitabine and albumin-bound paclitaxel, and a concomitant drug of four drugs (oxaliplatin, levofolinate, irinotecan, and fluorouracil).

The present disclosure also provides a pharmaceutical composition for treating a disease, comprising the antibody or the fragment thereof of the present disclosure. Specifically, for example, the present disclosure further provides a pharmaceutical composition for treating a disease, comprising the antibody or the fragment thereof of the present disclosure labeled with, for example, various anticancer agents, toxin compounds, such as lysine, and radioactive substances, such as radioactive metal ions (for example, compounds having radioactive activity, such as alpha emitter). Examples of the method for labeling include a method of indirectly binding a compound using a linker, such as polyethylene glycol, and a method of directly binding a compound by forming a disulfide bond at a cysteine residue of the antibody or the fragment thereof. It is also possible to directly bind a compound to the antibody or the fragment thereof using a heterologous bifunctional crosslinking agent, such as N-succinyl-3-(2-pyridyldithio)propionate. Furthermore, a compound can also be bound by oxidizing the sugar chain in the Fc region of the antibody.

The pharmaceutical composition for treating a disease of the present disclosure can further comprise a pharmaceutically acceptable carrier. Examples of usable pharmaceutically acceptable carriers include standard pharmaceutical carriers known in the art, such as phosphate-buffered saline solutions, water, oil/water emulsions and other emulsions, and various types of wetting agents. In addition, other components, such as a pharmaceutical-grade stabilizer, buffer, preservative, and excipient, can also be used in the pharmaceutical composition for treating a disease of the present disclosure, and preparation of the pharmaceutical composition in consideration of pH, isotonicity, stability, etc. can be carried out by a known method.

The dose of the antibody or the fragment thereof contained in the pharmaceutical composition for treating a disease of the present disclosure and the dose of the antibody or the fragment thereof labeled with the therapeutic agent are not particularly limited as long as each of the doses is a pharmacologically effective amount. Each of the doses can be determined as appropriate according to race, gender, age, type of cancer, etc., and each of the antibodies or the fragments thereof can usually be administered at 0.01 to 1,000 mg/kg and preferably at 0.1 to 100 mg/kg once every 1 to 180 days, or twice or three times or more a day.

Examples of diseases that can be treated using the pharmaceutical composition of the present disclosure include cancers, such as the pancreatic cancer described above, and the pharmaceutical composition of the present disclosure is useful for treating a cancer that highly expresses a molecule to which the antibody or the fragment thereof of the present disclosure binds. That is, when it is determined by the test method of the present disclosure that the possibility of having a cancer, such as pancreatic cancer, in the subject is high, it is possible to treat a cancer, such as pancreatic cancer, in the subject by administering the pharmaceutical composition of the present disclosure.

The present disclosure also provides a pharmaceutical composition for inhibiting metastasis of cancer, comprising the antibody or the fragment thereof of the present disclosure. The configuration and application of this pharmaceutical composition are the same as those of the pharmaceutical composition described above.

### EXAMPLES

The present invention is described in detail below based on Examples; however, the present invention is not limited to these Examples.

### Example 1: Production of Rabbit Monoclonal Antibodies that Bind to Extracellular Vesicles Derived from Pancreatic Cancer Cells

### (a) Preparation of Lymphocytes

Frozen human early-stage pancreatic cancer tissue (ProteoGenex, stage 1, tumor occupancy rate: 85%, 55-year-old female) was chopped and suspended in PBS to 0.06 mg/mL to obtain an immunization antigen. JW/CSK rabbits (13 weeks old, female) were immunized with an antigen solution containing a mixture of the immunization antigen and FCA. Three weeks later, enlarged lymph nodes were collected, and lymphocytes obtained by homogenization and treatment with a hemolytic agent were frozen in a cryoprotective medium (Cellbanker 1, Nippon Zenyaku Kogyo Co., Ltd.).

### (b) Preparation of Extracellular Vesicles

At the Osaka International Cancer Institute, tumor tissue was surgically removed from patients with early-stage pancreatic cancer with tumors of 2 cm or less. The tumor tissue was washed with antibiotic-containing saline and then chopped into small pieces of about 1 mm. A DMEM medium containing 1 mg/mL collagenase (Sigma-Aldrich, catalog number: 044-29765) was added thereto, followed by shaking (200 rpm, 37° C., 15 minutes). Subsequently, tumor cells derived from the tumor tissue were collected through a 200 μm mesh. The tumor cells were cultured in a DMEM medium containing 10 ng/mL basic FGF and 2 ng/mL TGF-β, and the culture supernatant was collected every 2 to 4 days and stored at −80° C. while fresh medium was added to the tumor cells to continue the culture.

The culture supernatant stored at −80° C. was thawed and then centrifuged (300×g, 4° C., 5 minutes; 1,200×g, 4° C., 20 minutes; 10,000×g, 4° C., 30 minutes) to collect the supernatant. From the collected supernatant, extracellular vesicles were isolated according to the protocol using a MagCapture Exosome Isolation Kit PS (FUJIFILM Wako Pure Chemical Corporation, catalog number: 290-84103). The protein concentration of the extracellular vesicles was quantified by using a BCA protein assay kit (Thermo Fisher, catalog number: 23225) with a BSA solution as the standard.

(c) Selection of Lymphocytes that Produce Antibodies that Bind to Extracellular Vesicles

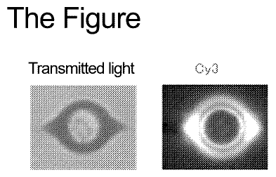

The extracellular vesicles of (b) were immobilized in a micro-chamber with 196,000 wells, each having a diameter of 20 μm. After washing, a 1% BSA solution was added for blocking at room temperature for 30 minutes. After removing the blocking solution, a mixture solution of the lymphocytes of (a) (2×10⁵ cells) and Cy3-labeled donkey anti-rabbit IgG (H+L) (Jackson ImmunoResearch Inc., catalog number: 711-165-152) was added and cultured for 45 minutes. The micro-chamber was then placed in an AS ONE Cell Picking System (AS ONE Corporation), and lymphocytes that produce antibodies that bind to the extracellular vesicles were selected. As a result, as shown in FIG. 1, clone PEMb14 that produces an antibody that reacts with the extracellular vesicles secreted by pancreatic cancer cells from the patients with early-stage pancreatic cancer was obtained.

### (d) Determination of PEMb14 Antibody Gene

After isolating clone PEMb14 with the AS ONE Cell Picking System, the antibody gene of the lymphocytes was amplified by single-cell PCR (Invitrogen, catalog number: 12574-035). The amplified gene fragments of the heavy chain and the light chain were each inserted into pCEC3.2 rabbit vector (Cell Engineering Corporation) for cloning.

The CDR base sequences of the heavy chain and the light chain of the PEMb14 antibody were determined using a DNA sequencer, and the amino acid sequences of the heavy-chain CDRs 1-3 and the light-chain CDRs 1-3 shown in Table 1, and the base sequences of the heavy-chain CDRs 1-3 and the light-chain CDRs 1-3 shown in Table 2 were identified. The amino acid sequences and base sequences of the entire heavy-chain variable region and light-chain variable region are shown below.

TABLE 1

| SEQ | | Amino acid |
|---|---|---|
| ID NO: | Sequence name | sequence |
| 1 | Heavy-chain CDR1 | EFSFSSSYY |
| 2 | Heavy-chain CDR2 | LYAGSGGVT |
| 3 | Heavy-chain CDR3 | AREVPADAAYGYFNL |
| 4 | Light-chain CDR1 | QSVYNNNN |
| | Light-chain CDR2 | SAS |
| 5 | Light-chain CDR3 | LGDFGGGIRA |

TABLE 2

| SEQ | | |
|---|---|---|
| ID NO: | Sequence name | Base sequence |
| 7 | Heavy-chain CDR1 | GAATTCTCCTTCAGTAGCAGCTACTAC |
| 8 | Heavy-chain CDR2 | CTTTATGCTGGTAGTGGTGGTGTCACT |
| 9 | Heavy-chain CDR3 | GCGAGAGAGGTCCCTGCTGATGCTGC |
| | | TTATGGATACTTTAACTTA |
| 10 | Light-chain CDR1 | CAGAGTGTTTATAATAACAACAAC |
| | Light-chain CDR2 | TCTGCATCC |
| 12 | Light-chain CDR3 | CTAGGCGATTTTGGTGGTGGTATCCGG |
| | | GCT |

Heavy-chain variable region (amino acid sequence)
(SEQ ID NO: 13)
METGLRWLLLVAVLKGVQCEQLEESGGDLVKPGASLTLTCTASEFSFSSSYYMCWVRQAPGKGL
EWIACLYAGSGGVTYYASWAKGRFTISKTSSTTVTLQMTSLTAADTATYFCAREVPADAAYGYF.
Light-chain variable region (amino acid sequence)
(SEQ ID NO: 14)
MDTRAPTQLLGLLLLWLPGATIAQVLTQTPSPVSAAVGGTVTINCQASQSVYNNNNLAWFQQKP
GQPPKQLIYSASTLASGVSSRFKGSGSGTQFTLTISGVQCDDAATYYCLGDFGGGIRA.
Heavy-chain variable region (base sequence)
(SEQ ID NO: 15)
ATGGAGACTGGGCTGCGCTGGCTTCTCCTGGTCGCTGTGCTCAAAGGTGTCCAGTGTGAGCAGC
TGGAGGAGTCCGGGGGAGACCTGGTCAAGCCTGGGGCATCCCTGACACTCACCTGCACAGCCTC
TGAATTCTCCTTCAGTAGCAGCTACTACATGTGCTGGGTCCGCCAGGCTCCAGGGAAGGGGCTG
GAGTGGATCGCTTGCCTTTATGCTGGTAGTGGTGGTGTCACTTACTACGCGAGCTGGGCGAAAG
GCCGATTCACCATCTCCAAAACCTCGTCGACCACGGTGACTCTGCAAATGACCAGTCTGACAGC
CGCGGACACGGCCACCTATTTCTGTGCGAGAGAGGTCCCTGCTGATGCTGCTTATGGATACTTT
AACTTA.
Light-chain variable region (base sequence)
(SEQ ID NO: 16)
ATGGACACGAGGGCCCCCACTCAGCTGCTGGGGCTCCTGCTGCTCTGGCTCCCAGGTGCCACAA
TTGCTCAAGTGCTGACCCAGACTCCATCCCCTGTGTCTGCCGCTGTGGGAGGCACAGTCACCAT
CAATTGCCAGGCCAGTCAGAGTGTTTATAATAACAACAACTTAGCCTGGTTTCAGCAGAAACCA
GGGCAGCCTCCCAAGCAACTGATCTATTCTGCATCCACTCTGGCATCTGGGGTGTCATCGCGGT
TCAAAGGCAGTGGATCTGGGACACAGTTCACTCTCACCATCAGCGGCGTGCAGTGTGACGATGC
TGCCACTTATTACTGTCTAGGCGATTTTGGTGGTGGTATCCGGGCT.

### (e) Preparation of PEMb14 Antibody (Rabbit IgG, κ)

Thirty 100 mm culture plates containing HEK293 cells (1.2×10⁷ cells per plate) were prepared, and the 2 types of vectors for expression of rabbit antibody (IgG) (heavy chain and light chain) obtained in Example 1(d) were transfected at 8.50 μg each per plate using PEI (Merck, catalog number: 408727-100ML), followed by culture for 7 days, and the culture supernatant containing the PEMb14 antibody was collected. The obtained culture supernatant was subjected to Ab-Capcher (ProteNova, catalog number: P-002-200), washed with TBS buffer, and then eluted with Gentle Ag/Ab Elution buffer (pH: 6.6) (Thermo Fisher, catalog number: #21027). Next, the resulting product was dialyzed against TBS buffer containing 0.2 M arginine, PBS buffer containing 0.2 M arginine, and PBS buffer, in this order. The antibody concentration was determined with a BCA Protein Assay Kit (Thermo Fisher Scientific, catalog number: 23225).

### Example 2: Blood Test for Patients with Early-Stage Pancreatic Cancer Using PEMb14 Antibody

### (a) Peroxidase Labeling of PEMb14 Antibody

10 μg of the rat anti-human CD63 monoclonal antibody (Cell Engineering Corporation, clone name: 1C8-2B11) was peroxidase-labeled according to the protocol using an Ab-10 Rapid Peroxidase Labeling Kit (Dojindo Laboratories, catalog number: LK33).

### (b) Preparation of Serum Samples

As biological samples, serum was collected at the Osaka International Cancer Institute, and CA19-9 levels were measured. The pancreatic cancer stage was determined according to the UICC TNM classification (8th edition, 2017). Each serum was diluted 10-fold with PBS to prepare serum samples.

### (c) Measurement of Antigen Value of PEMb14 Antibody in the Serum of Healthy Subjects and Pancreatic Cancer Patients

100 μl of 10 μg/ml PEMb14 antibody (rabbit IgG) was added to each well of a MaxiSorp 96-well plate, and the plate was allowed to stand at room temperature for 1 hour. Next, each well was washed 3 times with 200 μl of a washing liquid (50 mM Tris-HCl (pH: 8.0) containing 0.05% Triton-X 100, 140 mM NaCl), and 200 μl of a blocking liquid (a washing liquid containing 5% BSA) was added thereto, and the plate was allowed to stand at room temperature for 1 hour. The blocking liquid was removed, and 100 μl of the serum sample was added and reacted with shaking at room temperature for 2 hours. Thereafter, each well was washed 5 times. Next, 100 μl of peroxidase-labeled rat anti-human CD63 monoclonal antibody diluted 1,000-fold with a blocking liquid was added to each well and the plate was allowed to stand at room temperature for 1 hour. After each well was washed 5 times, 100 μl of a peroxidase substrate solution (SeraCare Life Sciences, code number: 5120-0053) was added to each well and reacted with shaking at room temperature for 20 minutes. After the reaction, 50 μl of 2% sulfuric acid was added to stop the reaction, and the absorbance at 450 nm in each well was measured using a microplate reader.

As a result, as shown in Table 3, the patients with pancreatic cancer, in particular, early-stage pancreatic cancer (stage 0 and stage 1) had a positive rate based on the antigen value of the PEMb14 antibody in the serum higher than the CA19-9 positive rate. These results indicate that determining the possibility of having pancreatic cancer, in particular, early-stage pancreatic cancer is possible based on the antigen value of the PEMb14 antibody.

TABLE 3

| | Number of | PEMb14/number | CA19-9/number |
|---|---|---|---|
| | serum | of positives | of positives |
| Stage | samples | (positive rate) | (positive rate) |
| 0 | 5 | 3 (60%) | 0 (0%) |
| 0 | 8 | 5 (63%) | 5 (63%) |
| 0 + 1 | 13 | 8 (62%) | 5 (38%) |

### Comparative Example 1: Production of Rabbit Monoclonal Antibodies that Bind to Extracellular Vesicles Derived from the Culture Supernatant of Pancreatic Cancer Cell Lines

(a) Preparation of Extracellular Vesicles Derived from the Culture Supernatant of Pancreatic Cancer Cell Lines

Commercially available human pancreatic cancer cell lines (KP-3, KP-3L, BxPC3, Mia-PaCa2, and SUIT-2) were each cultured in a 10% FBS-containing RPMI 1640 medium for 3 days, and the culture supernatants were collected. From the collected supernatants, extracellular vesicles were isolated according to the protocol using a MagCapture Exosome Isolation Kit PS, and the 5 types of obtained extracellular vesicles were then mixed. The protein concentration of the extracellular vesicles was quantified using a BCA protein assay kit with a BSA solution as the standard.

(b) Selection of Lymphocytes that Produce Antibodies that Bind to Extracellular Vesicles

In the same manner as in Example 1(c), the extracellular vesicles of (a) were immobilized in a micro-chamber, the lymphocytes of Example 1(a) (2×10⁵ cells) were added, and lymphocytes that produce antibodies that bind to the extracellular vesicles were selected. As a result, 6 different clones that produce antibodies that react with the extracellular vesicles secreted by the pancreatic cancer cell lines were obtained. Next, antibodies (d5, d13, d15, d17, d19, d29) were prepared from each clone in the same manner as in Example 1(e).

### (c) Measurement of Antigen Value in the Serum of Healthy Subjects and Pancreatic Cancer Patients

As biological samples, the serum (Proteogenex, 13 healthy subjects and 15 pancreatic cancer (stage 1) patients) was diluted 10-fold with PBS to prepare serum samples. The antigen values were determined in the same manner as in Example 2 (c).

As a result, as shown in Table 4, the patients with early-stage pancreatic cancer (stage 1) had a positive rate based on each antigen value in the serum lower than that of PEMb14. These results indicate that an antibody that has an ability to bind to extracellular vesicles derived from a medium obtained by culturing at least one biological sample selected from the group consisting of biological tissues and cells isolated from the biological tissues is more useful than an antibody that has an ability to bind to extracellular vesicles derived from a medium obtained by culturing artificially established cell lines.

TABLE 4

| Antibody | Pancreatic cancer | Number of serum | Number of positives | |
|---|---|---|---|---|
| name | stage | samples | (positive rate) | |
| d5 | 1 | 15 | 1 | (7%) |
| d13 | 1 | 15 | 1 | (7%) |
| d15 | 1 | 15 | 0 | (0%) |
| d17 | 1 | 15 | 2 | (13%) |
| d19 | 1 | 15 | 3 | (20%) |
| d29 | 1 | 15 | 0 | (0%) |

### Comparative Example 2: Production of Rabbit Monoclonal Antibodies that Bind to Extracellular Vesicles Derived from the Serum of Pancreatic Cancer Patients

(a) Preparation of Extracellular Vesicles Derived from the Serum of Pancreatic Cancer Patients

Extracellular vesicles were isolated from 1 mL each of the serum of 10 pancreatic cancer patients (stage 1) (Proteogenex, 3 males and 7 females, 40 to 65 years old) according to the protocol using a MagCapture Exosome Isolation Kit PS, and the 10 types of obtained extracellular vesicles were then mixed. The protein concentration of the extracellular vesicles was quantified using a BCA protein assay kit with a BSA solution as the standard.

(b) Selection of Lymphocytes that Produce Antibodies that Bind to Extracellular Vesicles

In the same manner as in Example 1(c), the extracellular vesicles of (a) were immobilized in a micro-chamber, the lymphocytes of Example 1(a) (2×10⁵ cells) were added, and lymphocytes that produce antibodies that bind to the extracellular vesicles were selected. As a result, 5 different clones that produce antibodies that react with the extracellular vesicles contained in the serum of the patients with early-stage pancreatic cancer (stage 1) were obtained. Next, antibodies (hs8, hs15, hs22, hs28, hs39) were prepared from each clone in the same manner as in Example 1(e).

### (c) Measurement of Antigen Values in the Serum of Healthy Subjects and Pancreatic Cancer Patients

As biological samples, the serum (Proteogenex, 13 healthy subjects and 15 pancreatic cancer (stage 1) patients) was diluted 10-fold with PBS to prepare serum samples. The antigen values were determined in the same manner as in Example 2(c).

As a result, as shown in Table 5, the patients with early-stage pancreatic cancer (stage 1) had a positive rate based on each antigen value in the serum lower than that of PEMb14. These results indicate that an antibody that has an ability to bind to extracellular vesicles derived from a medium obtained by culturing at least one biological sample selected from the group consisting of biological tissues and cells isolated from the biological tissues is more useful than an antibody that has an ability to bind to extracellular vesicles derived from the serum of pancreatic cancer patients.

TABLE 5

| Antibody | Pancreatic cancer | Number of serum | Number of positives |
|---|---|---|---|
| name | stage | samples | (positive rate) |
| hs8 | 1 | 15 | 2 (13%) |
| hs15 | 1 | 15 | 0 (0%) |
| hs22 | 1 | 15 | 0 (0%) |
| hs28 | 1 | 15 | 1 (7%) |
| hs39 | 1 | 15 | 0 (0%) |

1. An antibody or an antigen-binding fragment thereof, comprising
a heavy-chain CDR1 comprising the amino acid sequence of SEQ ID NO: 1,
a heavy-chain CDR2 comprising the amino acid sequence of SEQ ID NO: 2,
a heavy-chain CDR3 comprising the amino acid sequence of SEQ ID NO: 3,
a light-chain CDR1 comprising the amino acid sequence of SEQ ID NO: 4,
a light-chain CDR2 comprising the amino acid sequence represented by SAS, and
a light-chain CDR3 comprising the amino acid sequence of SEQ ID NO: 6.

2. The antibody or the antigen-binding fragment thereof according to claim 1, wherein the antibody is a monoclonal antibody.

3. The antibody or the antigen-binding fragment thereof according to claim 1, wherein the antibody is IgG.

4. The antibody or the antigen-binding fragment thereof according to claim 1, wherein the antigen-binding fragment is Fab, F(ab′)₂, a minibody, scFv-Fc, Fv, scFv, a diabody, a triabody, or a tetrabody.

5. A polynucleotide comprising a coding sequence of the antibody or the antigen-binding fragment thereof of claim 1.

6. A cell comprising the polynucleotide of claim 5.

7. A test drug for cancer, comprising the antibody or the antigen-binding fragment thereof of claim 1.

8. The test drug according to claim 7, wherein the cancer is metastatic cancer.

9. The test drug according to claim 7, wherein the cancer is pancreatic cancer.

10. The test drug according to claim 7, wherein the cancer is early-stage pancreatic cancer.

11. A pharmaceutical composition for diagnosing cancer, comprising the antibody or the antigen-binding fragment thereof of claim 1 labeled with a diagnostic agent.

12. The pharmaceutical composition according to claim 11, wherein the diagnostic agent is a radioactive substance.

13. The pharmaceutical composition according to claim 11, wherein the cancer is pancreatic cancer.

14. A pharmaceutical composition for treating cancer, comprising the antibody or the antigen-binding fragment thereof of claim 1.

15. The pharmaceutical composition for treating cancer according to claim 14, wherein the cancer is pancreatic cancer.

16. A pharmaceutical composition for inhibiting metastasis of cancer, comprising the antibody or the antigen-binding fragment thereof of claim 1.

17. The pharmaceutical composition for inhibiting metastasis of cancer according to claim 16, wherein the cancer is pancreatic cancer.


SEQUENCE LISTING (16 sequences)

SEQ ID NO: 1 - protein, 9 aa, synthetic construct
  REGION 1..9: note = H CDR1
EFSFSSSYY

SEQ ID NO: 2 - protein, 9 aa, synthetic construct
  REGION 1..9: note = H CDR2
LYAGSGGVT

SEQ ID NO: 3 - protein, 15 aa, synthetic construct
  REGION 1..15: note = H CDR3
AREVPADAAYGYFNL

SEQ ID NO: 4 - protein, 8 aa, synthetic construct
  REGION 1..8: note = L CDR1
QSVYNNNN

SEQ ID NO: 5 - skipped

SEQ ID NO: 6 - protein, 10 aa, synthetic construct
  REGION 1..10: note = L CDR3
LGDFGGGIRA

SEQ ID NO: 7 - DNA, 27 bp, synthetic construct
  misc_feature 1..27: note = H CDR1
  source 1..27: mol_type = other DNA
gaattctccttcagtagcagctactac

SEQ ID NO: 8 - DNA, 27 bp, synthetic construct
  misc_feature 1..27: note = H CDR2
  source 1..27: mol_type = other DNA
ctttatgctggtagtggtggtgtcact

SEQ ID NO: 9 - DNA, 45 bp, synthetic construct
  misc_feature 1..45: note = H CDR3
  source 1..45: mol_type = other DNA
gcgagagaggtccctgctgatgctgcttatggatactttaactta

SEQ ID NO: 10 - DNA, 24 bp, synthetic construct
  misc_feature 1..24: note = L CDR1
  source 1..24: mol_type = other DNA
cagagtgtttataataacaacaac

SEQ ID NO: 11 - skipped

SEQ ID NO: 12 - DNA, 30 bp, synthetic construct
  misc_feature 1..30: note = L CDR3
  source 1..30: mol_type = other DNA
ctaggcgattttggtggtggtatccgggct

SEQ ID NO: 13 - protein, 130 aa, synthetic construct
  REGION 1..130: note = H variable region
METGLRWLLLVAVLKGVQCEQLEESGGDLVKPGASLTLTCTASEFSFSSSYYMCWVRQAPGKGLEWIACLYAGSGGVTYYASWAKGRFTISKTSSTTVTLQMTSLTAADTATYFCAREVPADAAYGYFNL

SEQ ID NO: 14 - protein, 122 aa, synthetic construct
  REGION 1..122: note = L variable region
MDTRAPTQLLGLLLLWLPGATIAQVLTQTPSPVSAAVGGTVTINCQASQSVYNNNNLAWFQQKPGQPPKQLIYSASTLASGVSSRFKGSGSGTQFTLTISGVQCDDAATYYCLGDFGGGIRA

SEQ ID NO: 15 - DNA, 390 bp, synthetic construct
  misc_feature 1..390: note = H variable region
  source 1..390: mol_type = other DNA
atggagactgggctgcgctggcttctcctggtcgctgtgctcaaaggtgtccagtgtgagcagctggaggagtccgggggagacctggtcaagcctggggcatccctgacactcacctgcacagcctctgaattctccttcagtagcagctactacatgtgctgggtccgccaggctccagggaaggggctggagtggatcgcttgcctttatgctggtagtggtggtgtcacttactacgcgagctgggcgaaaggccgattcaccatctccaaaacctcgtcgaccacggtgactctgcaaatgaccagtctgacagccgcggacacggccacctatttctgtgcgagagaggtccctgctgatgctgcttatggatactttaactta

SEQ ID NO: 16 - DNA, 366 bp, synthetic construct
  misc_feature 1..366: note = L variable region
  source 1..366: mol_type = other DNA
atggacacgagggcccccactcagctgctggggctcctgctgctctggctcccaggtgccacaattgctcaagtgctgacccagactccatcccctgtgtctgccgctgtgggaggcacagtcaccatcaattgccaggccagtcagagtgtttataataacaacaacttagcctggtttcagcagaaaccagggcagcctcccaagcaactgatctattctgcatccactctggcatctggggtgtcatcgcggttcaaaggcagtggatctgggacacagttcactctcaccatcagcggcgtgcagtgtgacgatgctgccacttattactgtctaggcgattttggtggtggtatccgggct